(19) United States
(12) Patent Application Publication
(10) Pub. No.: US 2018/0256786 A1
(43) Pub. Date: Sep. 13, 2018

(54) BALLOON CATHETERS COMPRISING NON-LATEX ELASTOMERIC COMPOSITIONS AND PROCESSES FOR MANUFACTURING THE SAME

(71) Applicant: Edwards Lifesciences Corporation, Irvine, CA (US)

(72) Inventors: Jeong Soo Lee, Diamond Bar, CA (US); Carlos Juan Ayala Borrero, Irvine, CA (US)

(73) Assignee: Edwards Lifesciences Corporation, Irvine, CA (US)

(21) Appl. No.: 15/914,902

(22) Filed: Mar. 7, 2018

Related U.S. Application Data
(60) Provisional application No. 62/467,922, filed on Mar. 7, 2017.

(51) Int. Cl. A61L 29/04; C08L 53/02; A61L 29/06; A61M 25/10; B29C 47/00; B29C 65/00; B29C 65/02; B29C 65/48; B29C 70/30

(52) U.S. Cl. CPC A61L 29/049; B29K 2009/06; A61L 29/06; A61M 25/1029; B29C 47/0064; B29C 47/0066; B29C 66/344; B29C 47/0004; B29C 65/02; B29C 65/48; B29C 70/30; C08L 2205/025; C08L 2205/03; C08L 2203/02; A61M 2025/1031; B29C 2793/0027; B29C 2793/009; C08L 53/02

(57) ABSTRACT

A medical device comprising: an inflatable balloon comprising an elastomeric composition having a Shore A hardness of less than 35. The elastomeric composition comprises one or more thermoplastic elastomers. The one or more thermoplastic elastomers can be one or a combination of SEBS, SIS, and SIBS. The elastomeric composition can exclude latex materials, natural rubbers, thermoplastic polyolefin elastomers, thermoplastic vulcanates, and thermoplastic polyurethanes, thermoplastic polyester, thermoplastic co-polyester, thermoplastic polyamides, and thermoplastic co-polyamides.

### CROSS-REFERENCE TO RELATED APPLICATIONS

This application claims the benefit under 35 U.S.C. 119(e) of U.S. Provisional Patent Application No. 62/467,922 filed Mar. 7, 2017, the entire contents of which are incorporated by reference as if fully set forth herein.

### FIELD OF THE INVENTION

The invention relates to balloon catheters that can be used in a variety of medical devices and, more particularly, to non-latex balloon catheters.

### BACKGROUND

Balloon catheters are commonly employed in a number of different medical devices and surgical procedures. Typically, these devices comprise a catheter that can be guided through a body conduit in a patient and an inflatable balloon located at the distal end of the catheter. Inflation of the balloon is typically accomplished by filling the balloon with air or fluid to a desired degree of expansion. Once the procedure is completed, the balloon is deflated and withdrawn.

The Swan-Ganz balloon catheter is one example of a balloon catheter. It was introduced in the 1970s and is inserted into the pulmonary artery to monitor the heart's function, blood flow and blood pressure in and around the heart. Initially developed for the management of acute myocardial infarction, it gained widespread use in the management of a variety of critical illnesses and surgical procedures.

The inflatable balloons in the balloon catheters are typically made from natural rubber latex. Balloons made from natural rubber latex have desirable mechanical and physical characteristics. One such characteristic is its volume expansion per unit pressure. Latex balloons are able to achieve a relatively larger volume expansion over lower pressures as compared to other non-latex materials.

There are, however, many disadvantages associated with the use of natural rubber latex for balloon catheters.

One significant disadvantage is that it carries the risk of causing an anaphylactic reaction in people who are allergic to latex. This life-threatening reaction caused by the natural proteins or allergens present in natural rubber latex.

Another disadvantage is the numerous process steps required for manufacturing the inflatable balloons using natural rubber latex, including preparing a latex dispersion or emulsion, dipping a former in the shape of the article to be manufactured into the latex and curing or vulcanizing the latex while on the former, followed by removal of the formed balloon.

Accordingly, there exists a need in the medical device field for improved manufacturing processes for making synthetic elastomeric articles, such as balloon catheters. Especially desirable would be processes, which can produce synthetic elastomeric articles, which possess properties similar or superior to natural latex rubber, while at the same time permitting for cost-effective manufacturing.

### BRIEF SUMMARY

Described herein are medical devices that comprise elastomeric compositions that can be used to fabricate, among other things, inflatable balloons or other parts that can be inserted into a patient's body cavity or conduit and for which a non-latex material is desirable. The elastomeric compositions described herein can impart a number of unique and desirable physical and mechanical qualities for inflatable balloons. Additionally, the elastomeric compositions described herein can provide for more simple and cost-effective methods for manufacturing inflatable balloons and for assembling the inflatable balloons onto delivery catheters.

In one embodiment, the medical device comprises an inflatable balloon comprising an elastomeric composition. The elastomeric composition can comprise one or more thermoplastic elastomers. The elastomeric composition can have a Shore A hardness of less than 35.

In a separate aspect, the elastomeric composition can consist of the one or more thermoplastic elastomers.

In another separate aspect, the one or more thermoplastic elastomers can be a styrene block polymer. The one or more thermoplastic elastomers can have a styrene content of 20 mol % or less.

In another separate aspect, the one or more thermoplastic elastomers can consist of a styrene-ethylene/butadiene-styrene elastomer (SEBS).

In another separate aspect, the one or more thermoplastic elastomers can comprise a styrene-ethylene/butadiene-styrene elastomer (SEBS).

In another separate aspect, the one or more thermoplastic elastomers can further comprises a styrene-isoprene-styrene (SIS).

In another separate aspect, the one or more thermoplastic elastomers can consists of 40 wt. % to 60 wt. % SIS and 60 wt. % to 40 wt. % SEBS.

In another separate aspect, the one or more thermoplastic elastomers can have a Shore A hardness of 30 or less.

In another separate aspect, the elastomeric composition can have an elongation at break of at least 500% according to ASTM D412.

In another separate aspect, the elastomeric composition can have a tensile stress at break of at least 400 psi according to ASTM D412.

In another separate aspect, the elastomeric composition can have a tensile modulus lower than 200 psi according to ASTM D638.

In another separate aspect, the tension set of the elastomeric composition is about 10% or less.

In another separate aspect, the elastomeric composition can exclude latex materials, natural rubbers, thermoplastic polyolefin elastomers, thermoplastic vulcanates, and thermoplastic polyurethanes, thermoplastic polyester, thermoplastic co-polyester, thermoplastic polyamides, and thermoplastic co-polyamides.

In another separate aspect, the inflatable balloon has a wall thickness of about 0.008 inches to about 0.025 inches.

In another separate aspect, the deployment pressure of the inflatable balloon is less than 10 psi.

In another separate aspect, the burst pressure of the inflatable balloon is greater than 18 psi.

In another embodiment, an elastomeric composition is provided. The elastomeric composition can comprise at least one strain-induced crystallizable polymer and at least one amorphous polymer. The elastomeric composition can have a Shore A hardness of 40 or less.

In a separate aspect, the at least one strain-induced crystallizable polymer and the at least one amorphous polymer can be styrene block polymers.

In another separate aspect, the at least one strain-induced crystallizable polymer can have a Shore A hardness of 30 or less.

In another separate aspect, wherein the at least one amorphous polymer can have a Shore A hardness of 30 or less.

In another separate aspect, the at least one strain-induced crystallizable polymer can be provided in the polymer blend in an amount of about 30% to about 50% by weight.

In another separate aspect, the at least one amorphous polymer can be provided in the polymer blend in an amount of about 50% to about 70% by weight.

In another separate aspect, the at least one strain-induced crystallizable polymer can be a styrene-ethylene/butadiene-styrene elastomer (SEBS).

In another separate aspect, the at least one amorphous polymer can be one or both of an SIS or SIBS.

In another separate aspect, the elastomeric composition can comprise 40 wt. % to 60 wt. % SIS and 60 wt. % to 40 wt. % SEBS.

In another separate aspect, the elastomeric composition can have an elongation at break of at least 500% according to ASTM D412.

In another separate aspect, the elastomeric composition can have a tensile stress at break of at least 400 psi according to ASTM D412.

In another separate aspect, the elastomeric composition can have a tensile modulus lower than 200 psi according to ASTM D638.

In another separate aspect, the elastomeric composition can exclude latex materials, natural rubbers, thermoplastic polyolefin elastomers, thermoplastic vulcanates, and thermoplastic polyurethanes, thermoplastic polyester, thermoplastic co-polyester, thermoplastic polyamides, and thermoplastic co-polyamides.

In another separate aspect, the elastomeric composition can have a Shore A hardness of 30 or less.

In a further embodiment, a medical device is provided. The medical device can comprise a delivery catheter and an inflatable balloon secured to the delivery catheter. The inflatable balloon comprises an elastomeric composition. The elastomeric composition can be provided in accordance with any of the foregoing aspects described above.

In yet a further embodiment, a method of manufacturing a balloon for a balloon catheter is provided. The method can comprise melting one or more thermoplastic elastomers, extruding the one or more thermoplastic elastomers to form an extruded tube, and mounting the extruded tube onto a catheter. The extruded tube can have a Shore A hardness of 40 or less.

In a separate aspect, the extruded tube can have a Shore A hardness of 30 or less.

In another separate aspect, the method can further comprise cutting the extruded tube to a desired length before the mounting.

In another separate aspect, the method can further comprise stretching the extruded tube during the mounting. The mounting can be achieved by heat, adhesive or by winding filaments over the extruded tube at proximal and distal ends of the extruded tube.

In another separate aspect, the method can exclude one or more of the following steps: dip molding, vulcanizing, blow molding, and annealing.

It is understood that each of the above-described aspects are separately provided and can also be provided in various combinations with one another, including such combinations not specifically described. Other objects, features and advantages of the described embodiments will become apparent to those skilled in the art from the following detailed description. It is to be understood, however, that the detailed description and specific examples, while indicating embodiments of the present invention, are given by way of illustration and not limitation. Many changes and modifications within the scope of the present invention can be made without departing from the spirit thereof, and the invention includes all such modifications.

### BRIEF DESCRIPTION OF THE DRAWINGS

Illustrative embodiments of the present disclosure are described herein with reference to the accompanying drawings, in which:

FIG. 1 depicts an embodiment of a balloon catheter with the balloon in an inflated state.

FIG. 2 is a perspective view of an extruded tube that can be assembled onto a catheter.

Like numerals refer to like parts throughout the several views of the drawings.

### DETAILED DESCRIPTION OF THE EXEMPLARY EMBODIMENTS

Specific, non-limiting embodiments of the present invention will now be described with reference to the drawings. It should be understood that such embodiments are by way of example and are merely illustrative of but a small number of embodiments within the scope of the present invention. Various changes and modifications obvious to one skilled in the art to which the present invention pertains are deemed to be within the spirit, scope and contemplation of the present invention as further defined in the appended claims.

Elastomeric Compositions.

The elastomeric compositions described herein can be used to fabricate inflatable balloons, among other things, that are commonly used in connection with balloon catheters. The elastomeric compositions can have a number of desirable physical characteristics that impart the desired mechanical characteristics onto the resulting inflatable balloon, including decreased bagginess of the balloon following inflation, deployment at low pressure, and a burst pressure that is similar to latex balloons.

In one embodiment, the elastomeric composition can comprises one or more thermoplastic elastomers.

In one embodiment, the one or more thermoplastic elastomers can comprise one or more styrene block polymers, hydrogenated styrene block copolymers, or functionalized analogues of styrene block copolymers, for example maleated styrene-ethylene/butadiene-styrene elastomer (SEBS Maleated). Exemplary styrene block polymers include styrene-ethylene/butadiene-styrene elastomer (SEBS), styrene-isoprene-styrene (SIS), or styrene-isoprene-butadiene-styrene (SIBS). In an aspect of this embodiment, the styrene block polymers can be any one of a SEBS or a combination of SEBS, SIS, and SIBS.

In one embodiment, the styrene block polymer can have a styrene content of about 40 mol % or less, about 35 mol % or less, about 30 mol % or less, about 25 mol % or less, about 20 mol % or less, about 15 mol % or less, about 10 mol % or less, or about 5 mol % or less. In one aspect of this embodiment, the styrene block polymer can have a styrene content in a range that includes and/or is between any two of the foregoing mol % values.

The elastomeric composition can consist solely of the one or more thermoplastic elastomers or the elastomeric composition can comprise the one or more thermoplastic elastomers and one or more additives. In one aspect, the one or more additives may be provided to alter the physical and/or mechanical properties of the elastomeric composition, which are desirable for the inflatable balloon.

In another embodiment, the one or more thermoplastic elastomers can comprise a combination of a strain-induced crystallizable polymer and an amorphous polymer. In one aspect of the embodiment, the strain-induced crystallizable polymer can be a SEBS and the amorphous polymer can be any one or a combination of SIS and SIBS.

In one embodiment, the elastomeric composition can consist solely of the one or more thermoplastic elastomers. In one aspect of this embodiment, the elastomeric composition can comprise one or a combination of SEBS, SIS and/or SIBS. In another aspect of this embodiment, the elastomeric composition can consist solely of one or a combination of SEBS, SIS, and/or SIBS.

In another embodiment, the elastomeric composition can comprise SEBS, SIS and/or SIBS in combination with one or more additional thermoplastic elastomers.

In one aspect, the elastomeric composition can comprise SEBS in combination with one or more thermoplastic elastomers different from SEBS. Accordingly, the SEBS can be provided in an amount of about 99 wt. % or less, about 98 wt. % or less, about 97 wt. % or less, about 96 wt. % or less, about 95 wt. % or less, about 94 wt. % or less, 93 wt. % or less, about 92 wt. % or less, about 91 wt. % or less, about 90 wt. % or less, about 89 wt. % or less, about 88 wt. % or less, about 87 wt. % or less, about 86 wt. % or less, about 85 wt. % or less, about 84 wt. % or less, about 83 wt. % or less, about 82 wt. % or less, about 81 wt. % or less, about 80 wt. % or less, about 79 wt. % or less, about 78 wt. % or less, about 77 wt. % or less, about 76 wt. % or less, about 75 wt. % or less, about 74 wt. % or less, about 73 wt. % or less, about 72 wt. % or less, about 71 wt. % or less, about 70 wt. % or less, about 69 wt. % or less, about 68 wt. % or less, about 67 wt. % or less, about 66 wt. % or less, about 65 wt. % or less, about 64 wt. % or less, about 63 wt. % or less, about 62 wt. % or less, about 61 wt. % or less, about 60 wt. % or less, about 59 wt. % or less, about 58 wt. % or less, about 57 wt. % or less, about 56 wt. % or less, about 55 wt. % or less, about 54 wt. % or less, about 53 wt. % or less, about 52 wt. % or less, about 51 wt. % or less, about 50 wt. % or less, about 49 wt. % or less, about 48 wt. % or less, about 47 wt. % or less, about 46 wt. % or less, about 45 wt. % or less, about 44 wt. % or less, about 43 wt. % or less, about 42 wt. % or less, about 41 wt. % or less, about 40 wt. % or less, about 39 wt. % or less, about 38 wt. % or less, about 37 wt. % or less, about 36 wt. % or less, about 35 wt. % or less, about 34 wt. % or less, about 33 wt. % or less, about 32 wt. % or less, about 31 wt. % or less, about 30 wt. % or less, about 29 wt. % or less, about 28 wt. % or less, about 27 wt. % or less, about 26 wt. % or less, about 25 wt. % or less, about 24 wt. % or less, about 23 wt. % or less, about 22 wt. % or less, about 21 wt. % or less, about 20 wt. % or less, about 19 wt. % or less, about 18 wt. % or less, about 17 wt. % or less, about 16 wt. % or less, about 15 wt. % or less, about 14 wt. % or less, about 13 wt. % or less, about 12 wt. % or less, about 11 wt. % or less, about 10 wt. % or less, about 9 wt. % or less, about 8 wt. % or less, about 7 wt. % or less, about 6 wt. % or less, about 5 wt. % or less of the elastomeric composition. In another aspect, the elastomeric composition can comprise an amount of the SEBS in the elastomeric composition in a range that includes and/or is between any two of the foregoing values. In one aspect, the one or more thermoplastic elastomers different from SEBS can constitute the remaining portion of the elastomeric composition to constitute 100 wt. %.

In another aspect, the elastomeric composition can comprise SIS and/or SIBS in combination with one or more thermoplastic elastomers different from SIS and SIBS. Accordingly, the SIS and/or SIS can be provided in an amount of about 99 wt. % or less, about 98 wt. % or less, about 97 wt. % or less, about 96 wt. % or less, about 95 wt. % or less, about 94 wt. % or less, 93 wt. % or less, about 92 wt. % or less, about 91 wt. % or less, about 90 wt. % or less, about 89 wt. % or less, about 88 wt. % or less, about 87 wt. % or less, about 86 wt. % or less, about 85 wt. % or less, about 84 wt. % or less, about 83 wt. % or less, about 82 wt. % or less, about 81 wt. % or less, about 80 wt. % or less, about 79 wt. % or less, about 78 wt. % or less, about 77 wt. % or less, about 76 wt. % or less, about 75 wt. % or less, about 74 wt. % or less, about 73 wt. % or less, about 72 wt. % or less, about 71 wt. % or less, about 70 wt. % or less, about 69 wt. % or less, about 68 wt. % or less, about 67 wt. % or less, about 66 wt. % or less, about 65 wt. % or less, about 64 wt. % or less, about 63 wt. % or less, about 62 wt. % or less, about 61 wt. % or less, about 60 wt. % or less, about 59 wt. % or less, about 58 wt. % or less, about 57 wt. % or less, about 56 wt. % or less, about 55 wt. % or less, about 54 wt. % or less, about 53 wt. % or less, about 52 wt. % or less, about 51 wt. % or less, about 50 wt. % or less, about 49 wt. % or less, about 48 wt. % or less, about 47 wt. % or less, about 46 wt. % or less, about 45 wt. % or less, about 44 wt. % or less, about 43 wt. % or less, about 42 wt. % or less, about 41 wt. % or less, about 40 wt. % or less, about 39 wt. % or less, about 38 wt. % or less, about 37 wt. % or less, about 36 wt. % or less, about 35 wt. % or less, about 34 wt. % or less, about 33 wt. % or less, about 32 wt. % or less, about 31 wt. % or less, about 30 wt. % or less, about 29 wt. % or less, about 28 wt. % or less, about 27 wt. % or less, about 26 wt. % or less, about 25 wt. % or less, about 24 wt. % or less, about 23 wt. % or less, about 22 wt. % or less, about 21 wt. % or less, about 20 wt. % or less, about 19 wt. % or less, about 18 wt. % or less, about 17 wt. % or less, about 16 wt. % or less, about 15 wt. % or less, about 14 wt. % or less, about 13 wt. % or less, about 12 wt. % or less, about 11 wt. % or less, about 10 wt. % or less, about 9 wt. % or less, about 8 wt. % or less, about 7 wt. % or less, about 6 wt. % or less, about 5 wt. % or less, about 4% or less, about 3% or less, or about 2% or less, about 1% or less of the elastomeric composition. In another aspect, the elastomeric composition can comprise an amount of the SIS and/or SIBS in the elastomeric composition in a range that includes and/or is between any two of the foregoing values. In one aspect, the one or more thermoplastic elastomers different from SIS and SIBS can constitute the remaining portion of the elastomeric composition to constitute 100 wt. %. In one embodiment, the elastomeric composition can consist of SEBS.

In one embodiment, the elastomeric composition can comprise or consist of a combination of SEBS and SIS, a combination of SEBS and SIBS or a combination or a combination of SEBS, SIS and SIBS.

In another embodiment, the elastomeric composition can exclude one, more than one, or all of the following materials: latex materials, natural rubbers, thermoplastic polyolefin elastomers, thermoplastic vulcanates, thermoplastic polyurethanes, thermoplastic polyester, thermoplastic co-polyester, thermoplastic polyamides, and/or thermoplastic co-polyamides.

Shore A Hardness.

The elastomeric compositions and the at least one thermoplastic elastomers can each characterized as having Shore A durometer hardnesses in accordance with the ASTM D2240-15e1, Standard Test Method for Rubber Property—Durometer Hardness, ASTM International, West Conshohocken, Pa., 2015, accessible at: https://www.astm.org/Standards/D2240.htm (hereinafter “ASTM D2240”).

In embodiments where the elastomeric composition consists of a single thermoplastic elastomer, it is understood that the Shore A hardness will be the same for both the elastomeric composition and the thermoplastic elastomers. In embodiments where the elastomeric composition comprises a combination of two or more thermoplastic elastomers, e.g. SEBS and SIS, it is understood that the Shore A hardness of the elastomeric composition can be different from one or both of the SEBS and SIS.

In an embodiment, the elastomeric composition or the one or more thermoplastic elastomers can each separately have a Shore A hardness of about 40 or less, about 39 or less, about 38 or less, about 37 or less, about 36 or less, about 35 or less, about 34 or less, about 33 or less, about 32 or less, about 31 or less, about 30 or less, about 29 or less, about 28 or less, about 27 or less, about 26 or less, about 25 or less, about 24 or less, about 23 or less, about 22 or less, about 21 or less, about 20 or less, about 19 or less, about 18 or less, about 17 or less, about 16 or less, about 15 or less, about 14 or less, about 13 or less, about 12 or less, about 11 or less, or about 10 or less. In one aspect of this embodiment, the elastomeric composition and the at least one thermoplastic elastomers can each separately can have a Shore A hardness in a range that includes and/or is between any two of the foregoing Shore A hardness values.

In one embodiment, the elastomeric composition can comprise or consist of one or more of a SEBS, SIS, or SIBS. As provided above, in one aspect of the embodiment, the SEBS, SIS or SIBS can each have a Shore A hardness of about 40 or less, about 39 or less, about 38 or less, about 37 or less, about 36 or less, about 35 or less, about 34 or less, about 33 or less, about 32 or less, about 31 or less, about 30 or less, about 29 or less, about 28 or less, about 27 or less, about 26 or less, about 25 or less, about 24 or less, about 23 or less, about 22 or less, about 21 or less, about 20 or less, about 19 or less, about 18 or less, about 17 or less, about 16 or less, about 15 or less, about 14 or less, about 13 or less, about 12 or less, about 11 or less, or about 10 or less. In one aspect of this embodiment, the SEBS can have a Shore A hardness in a range that includes and/or is between any two of the foregoing Shore A hardness values. Accordingly, the SEBS, SIS or SIBS can each have a Shore A hardness of about 10 to about 35.

In one embodiment, the elastomeric composition can comprise or consist of a SIS and/or SIBS. In one aspect of the embodiment, the SIS or SIBS can each have a Shore A hardness of about 40 or less, about 39 or less, about 38 or less, about 37 or less, about 36 or less, about 35 or less, about 34 or less, about 33 or less, about 32 or less, about 31 or less, about 30 or less, about 29 or less, about 28 or less, about 27 or less, about 26 or less, about 25 or less, about 24 or less, about 23 or less, about 22 or less, about 21 or less, about 20 or less, about 19 or less, about 18 or less, about 17 or less, about 16 or less, about 15 or less, about 14 or less, about 13 or less, about 12 or less, about 11 or less, or about 10 or less. In one aspect of this embodiment, the SIS or SIBS can each have a Shore A hardness in a range that includes and/or is between any two of the foregoing Shore A hardness values. Accordingly, the SIS or SIBS can have a Shore A hardness of about 10 to about 35.

Strain-Induced Crystallization.

The one or more thermoplastic elastomers is selected for the elastomeric composition such that it can undergo strain-induced crystallization. Strain-induced crystallization is a desirable characteristic because it provides important effects on strength and fatigue properties. There are various techniques for measuring crystallization, including x-ray diffraction, specific heat changes, and density changes. Crystallization can also be observed indirectly through its effects on the stress-strain fatigue behavior of the one or more thermoplastic elastomers. In one embodiment, the one or more thermoplastic elastomers is capable of undergoing strain crystallization such that balloons that are fabricated from the one or more thermoplastic elastomers can undergo a controlled inflation to the desired balloon diameter. Strain-induced crystallization can increase the modulus of the elastomer undergoing deformation thus limiting diameter growth of the balloon.

Plastic Deformation.

The one or more thermoplastic elastomers can be selected for the elastomeric composition such that the elastomeric composition can undergo a relatively low plastic deformation. Plastic deformation generally occurs when an elastomeric material does not revert to its previous dimension after it has been subjected to a form of stress. In this case, the inflatable balloons are subjected to inflation and expansion as a form of stress. Generally, the extent to which the inflatable balloon will undergo plastic deformation when inflated will determine the extent to which it will return to its original dimensions before inflation. Therefore, one desirable characteristic of an inflatable balloon is that it will exhibit relatively low or no plastic deformation such that the inflatable balloon is not “baggy” after inflation. In use, an inflatable balloon that is “baggy” runs the risk of tearing as it catches on, for example, a catheter introducer sheath. Thus, for inflatable balloons that exhibit significant plastic deformation, a larger introducer sheath may be required, which is often undesirable for catheters that are used in a patient's vasculature.

Accordingly, in one embodiment, the elastomeric composition exhibits a relatively low plastic deformation. This low plastic deformation can be determined by tensile or tension set as measured by ASTM D412. For example, an extruded tube that is 2 inches in length can be stretched to 11 inches in length and the difference in the stretched length and the original length can be expressed in a percentage. Thus, in this example, an extruded tube that returns to 2.2 inches after stretching would have a tensile set of 10%.

In one embodiment, the tensile set of the elastomeric composition or the one or more thermoplastic elastomers can be 20% or less, 15% or less, 10% or less, 5% or less, or 1%. The tensile set of the elastomeric composition can also be in a range that includes and/or is between any two of the foregoing values.

Elongation at Break.

The one or more thermoplastic elastomers can be selected for the elastomeric composition such that it has an elongation of break of at least about 400%, of at least about 425%, of at least about 450%, of at least about 500%, of at least about 525%, of at least about 550%, of at least about 575%, of at least about 600%, of at least about 625%, of at least about 650%, of at least about 675%, of at least about 700%, of at least about 725%, of at least about 750%, of at least about 775%, or at least about 800%. In one aspect of this invention, the elastomeric composition or the one or more thermoplastic elastomers can each separately have an elongation at break in a range that includes and/or is between any two of the foregoing values. The elongation at break of a material can be determined in accordance with ASTM D412.

Strain Break.

The one or more thermoplastic elastomers can be selected for the elastomeric composition such that the elastomeric composition has a strain break of at least about 400 psi, at least about 425 psi, at least about 450 psi, at least about 475 psi, at least about 500 psi, at least about 525 psi, at least about 550 psi, at least about 575 psi, at least about 600 psi, at least about 625 psi, at least about 650 psi, at least about 675 psi, at least about 700 psi, at least about 725 psi, at least about 750 psi, at least about 775 psi, at least about 800 psi, at least about 825 psi, at least about 850 psi, at least about 875 psi, at least about 900 psi, at least about 925 psi, at least about 950 psi, at least about 975 psi, or at least about 1,000 psi. In an aspect of this embodiment, the elastomeric composition can have a strain break in a range that includes and/or is between any two of the foregoing values.

Tensile Modulus.

A tensile modulus is a measure of an elastomeric material's resistance to elastic deformation, which occurs when a force is applied to a material that causes its shape to change. Once the force is removed, however, the elastomeric material returns to its original shape and size because elastic deformation is simply the stretching of atomic bonds. This is in contrast to plastic deformation, which occurs when the material does not return to its original shape and size even after the force is removed. Plastic deformation is permanent because the applied force is so great that the atoms actually move past each other. The tensile modulus is commonly measured by taking the amount of stress applied to a material and dividing it by the strain the material undergoes.

In an embodiment, the elastomeric composition or the one or more thermoplastic elastomers can have a low tensile modulus that permits for elastic deformation of the balloon when inflated. In one aspect of the embodiment, the tensile modulus of the elastomeric composition or the one or more thermoplastic elastomers can each separately be about 250 psi or lower, about 225 psi or lower, about 220 psi or lower, about 215 psi or lower, about 210 psi or lower, about 205 psi or lower, about 200 psi or lower, about 195 psi or lower, about 190 psi or lower, about 185 psi or lower, about 180 psi or lower, about 175 psi or lower, about 170 psi or lower, about 165 psi or lower, about 160 psi or lower, about 155 psi or lower, about 150 psi or lower, about 145 psi or lower, about 140 psi or lower, about 135 psi or lower, about 130 psi or lower, about 125 psi or lower, about 120 psi or lower, about 115 psi or lower, about 110 psi or lower, about 105 psi or lower, or about 100 psi or lower. In one aspect of this embodiment, the elastomeric composition or the one or more thermoplastic elastomers can each separately have a tensile modulus that is in a range that includes and is between any two of the foregoing values.

The tensile modulus can be determined in accordance with ASTM D638-14, Standard Test Method for Tensile Properties of Plastics, ASTM International, West Conshohocken, Pa., 2014, accessible at: http://www.astm.org/cgi-bin/resolver.cgi?D638-14 (hereinafter “ASTM D638”).

Inflatable Balloon Catheter.

The elastomeric compositions described above can be used to fabricate an inflatable balloon catheter. It is desirable to achieve the lowest delivery profile as practically possible since inflatable balloon catheters are inserted through a patient's vasculature. It is also desirable to have certain mechanical and physical characteristics for the balloon, such as an appropriate deployment pressure and burst pressure. Additionally, it is desirable to fabricate the insertable balloon catheter with materials that are non-antigenic. Natural rubber-based materials, such as latex, while having desirable mechanical and physical characteristics, have been known to cause allergic reactions in certain patients.

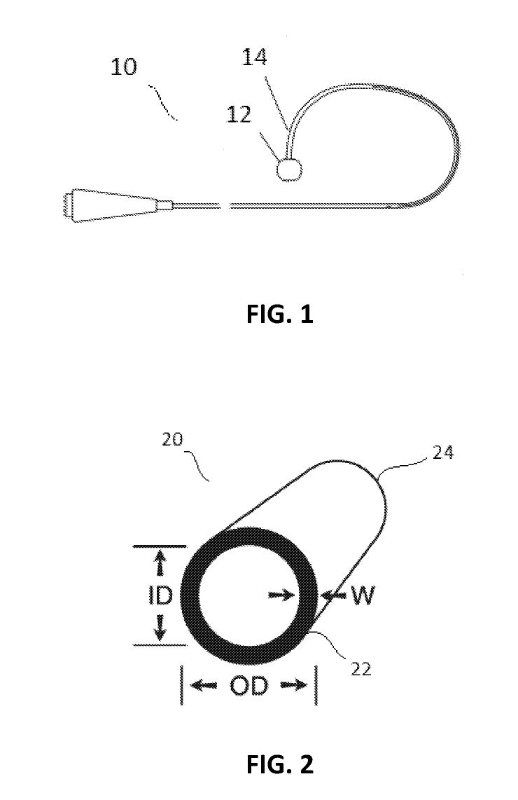

FIG. 1 depicts an exemplary inflatable balloon catheter (10) that can be made from the elastomeric compositions described herein. The inflatable balloon catheter (10) depicted in FIG. 1 is a pulmonary catheter, otherwise known as a Swan-Ganz catheter. It is understood that the inflatable balloon catheter (10) described in reference to FIG. 1 is an exemplary embodiment and that the invention is not limited to any particular type of inflatable balloon catheters.

As shown in FIG. 1, the balloon catheter (10) generally comprises an inflatable balloon (12) and a catheter (14). The inflatable balloon (12) may be constructed by extruding a tube of an elastomeric composition described herein. FIG. 2 depicts an exemplary tube (20) that can be mounted onto a catheter to form an inflatable balloon catheter. The extruded tube is then cut to the desired length and then mounted and secured on the catheter with an adhesive and primer.

Balloon Wall Thickness.

The wall thickness of the inflatable balloon can be chosen based on the application and, in certain instances, the profile that is desired in an uninflated state, the desired diameter of the inflatable balloon in the inflated state and the desired physical characteristics of the balloon, such as deployment pressure and burst pressure. As shown in FIG. 2, the inflatable balloon (10) can be formed from a tube having a wall thickness (W) in an uninflated state. This wall thickness (W) may further be reduced in the balloon in embodiments where the tube is stretched during mounting onto the catheter.

In an embodiment, the wall thickness of the inflatable balloon (uninflated), the extruded tube or the extruded tube in the stretched state can be about 0.005 inches, about 0.006 inches, about 0.007 inches, about 0.008 inches, about 0.009 inches, about 0.010 inches, about 0.011 inches, about 0.012 inches, about 0.013 inches, about 0.014 inches, about 0.015 inches, about 0.016 inches, about 0.017 inches, about 0.018 inches, about 0.019 inches, about 0.020 inches, about 0.021 inches, about 0.022 inches, about 0.023 inches, about 0.024 inches, about 0.025 inches, about 0.026 inches, about 0.027 inches, about 0.028 inches, about 0.029 inches, about 0.030 inches, about 0.031 inches, about 0.032 inches, about 0.033 inches, about 0.034 inches, about 0.035 inches, about 0.036 inches, about 0.037 inches, about 0.038 inches, about 0.039 inches, about 0.040 inches, about 0.041 inches, about 0.042 inches, about 0.043 inches, about 0.044 inches, about 0.045 inches, about 0.046 inches, about 0.047 inches, about 0.048 inches, about 0.049 inches, or about 0.050 inches. In one aspect of this embodiment, wall thickness can be provided in a range that includes and/or is between any two of the foregoing wall thickness values. In another aspect of this embodiment, the values reflect a single wall thickness. In a further aspect, the values reflect an average wall thickness of the uninflated balloon.

Deployment Pressure.

In an embodiment, the inflatable balloon that is constructed in accordance with the elastomeric compositions can be characterized as having a low and more controlled deployment pressure. In one embodiment, the deployment pressure can be ascertained when the internal volume of the inflatable balloon begins to increase (i.e., initial increase in volume) from its original non-inflated state. In one aspect of the embodiment, the deployment pressure at the initial increase in volume is about 20 psi or less, about 19 psi or less, about 18 psi or less, about 18 psi or less, about 17 psi or less, about 16 psi or less, about 15 psi or less, about 14 psi or less, about 13 psi or less, about 12 psi or less, about 11 psi or less, about 10 psi or less, about 9 psi or less, about 8 psi or less, about 7 psi or less, about 6 psi or less, or about 5 psi or less The deployment pressure can also be in a range that includes and/or is between any two of the foregoing values. The inflatable balloon that is made with latex exhibits a deployment pressure at about 9.5 psi. The inflatable balloons that are made with an elastomeric composition consisting solely of SEBS exhibits a deployment pressures of less than 7 psi, lower than balloon made from latex

Burst Pressure.

In an embodiment, the inflatable balloon can be characterized as having a similar or higher burst pressure in comparison to latex at same balloon wall thickness. The burst pressure of the inflatable balloon is the internal pressure of the inflatable balloon just prior to bursting. In one embodiment, the inflatable balloon can have a burst pressure of about 15 psi or greater, about 16 psi or greater, about 17 psi or greater, about 18 psi or greater, about 19 psi or greater, about 20 psi or greater, about 21 psi or greater, about 22 psi or greater, about 23 psi or greater, about 24 psi or greater, about 25 psi or greater, about 26 psi or greater, about 27 psi or greater, about 28 psi or greater, about 29 psi or greater, or about 30 psi or greater. The burst pressure can also be provided in a range that includes and/or is between any two of the foregoing values.

Manufacture and Assembly of the Inflatable Balloon Catheter.

Exemplary methods of manufacturing and assembling an inflatable balloon catheter are described herein.

In one embodiment, an elastomeric composition can have a desired Shore A hardness is provided. As described above, the elastomeric composition can consist or comprise of one or more thermoplastic elastomers. In one aspect of the embodiment, the elastomeric composition consists of SEBS and has a Shore A hardness of 30 or less. In another aspect of the embodiment, the elastomeric composition consists of SEBS and one or both of SIS and SIBS, and has a Shore A hardness of 30 or less.

The one or more thermoplastic elastomers can be provided as polymer pellets which are melted and extruded into a tube. The tube thickness can be provided in accordance with the balloon wall thickness described above. In embodiments where two or more thermoplastic elastomers are used, the polymer pellets can be combined before or during the extrusion. FIG. 2 depicts an extruded tube (20) having proximal (22) and distal (24) ends attachable to a delivery catheter. The extruded tube also comprises a desired outer diameter (OD), internal diameter (ID) and corresponding wall thickness (W=OD−ID).

The extruded tube can then be cut to a desired length to be mounted onto a delivery catheter, as shown in FIG. 1. The mounting can be achieved by placing the catheter body through the extruded tube and securing the proximal and distal ends to the catheter body using any one or a combination of applying heat and adhesives and primer. The extruded tube can also be mounted by winding a filament over the extruded tube at both proximal (22) and distal ends (24) of the extruded tube.

In one embodiment, the extruded tube can have a Shore A hardness of about 40 or less, about 39 or less, about 38 or less, about 37 or less, about 36 or less, about 35 or less, about 34 or less, about 33 or less, about 32 or less, about 31 or less, about 30 or less, about 29 or less, about 28 or less, about 27 or less, about 26 or less, about 25 or less, about 24 or less, about 23 or less, about 22 or less, about 21 or less, about 20 or less, about 19 or less, about 18 or less, about 17 or less, about 16 or less, about 15 or less, about 14 or less, about 13 or less, about 12 or less, about 11 or less, or about 10 or less. In one aspect of this embodiment, the extruded tube can have a Shore A hardness in a range that includes and/or is between any two of the foregoing Shore A hardness values.

In one embodiment, the extruded tube may be subjected to axial stretching before or during mounting on the catheter to reduce the wall thickness and plastic deformation further, if desired. In one aspect, the extruded tube can be stretched axially for 50% or more, 60% or more, 70% or more, 80% or more, 90% or more, 100% or more, 110% or more, 120% or more, 130% or more, 140% or more, 150% or more, 160% or more, 170% or more, 180% or more, 190% or more or 200% or more of its original length. The extruded tube can also be stretched axially within a range of percentages of its original length that includes and is between any two of the foregoing values. In one embodiment, the extruded tube is attached to the catheter while it is axially stretched. In other embodiment, the extruded tube is attached to the catheter after removing the forces causing the extruded tube to be axially stretched.

In another embodiment, the methods for manufacturing and assembling the inflatable balloon catheter excludes one or more of the following steps: dip molding, vulcanizing, blow molding, and annealing.

The non-limiting embodiments of the present invention described and claimed herein are not to be limited in scope by the specific embodiments disclosed herein, as these embodiments are intended as illustrations of several aspects of the invention. Indeed, various modifications of the invention in addition to those shown and described herein will become apparent to those skilled in the art from the foregoing description. Such modifications are also intended to fall within the scope of the appended claims.

1. A medical device comprising: an inflatable balloon comprising an elastomeric composition comprising one or more thermoplastic elastomers, the elastomeric composition having a Shore A hardness of less than 35.

2. The medical device of claim 1, wherein the elastomeric composition consists of the one or more thermoplastic elastomers.

3. The medical device of claim 1, wherein the one or more thermoplastic elastomers is a styrene block polymer.

4. The medical device of claim 3, wherein the one or more thermoplastic elastomers has a styrene content of 20 mol % or less.

5. The medical device of claim 1, wherein the one or more thermoplastic elastomers consists of a styrene-ethylene/butadiene-styrene elastomer (SEBS).

6. The medical device of claim 1, wherein the one or more thermoplastic elastomers comprises a styrene-ethylene/butadiene-styrene elastomer (SEBS).

7. The medical device of claim 6, wherein the one or more thermoplastic elastomers further comprises a styrene-isoprene-styrene (SIS).

8. The medical device of claim 7, wherein the one or more thermoplastic elastomers consists of: 40 wt. % to 60 wt. % SIS and 60 wt. % to 40 wt. % SEBS.

9. The medical device of claim 5, wherein the one or more thermoplastic elastomers has a Shore A hardness of 30 or less.

10. The medical device of claim 9, wherein the elastomeric composition has an elongation at break of at least 500% according to ASTM D412.

11. The medical device of claim 9, wherein the elastomeric composition has a tensile stress at break of at least 400 psi according to ASTM D412.

12. The medical device of claim 9, wherein the elastomeric composition has a tensile modulus lower than 200 psi according to ASTM D638.

13. The medical device of claim 9, wherein the tension set of the elastomeric composition is about 10% or less.

14. The medical device of claim 9, wherein the elastomeric composition excludes latex materials, natural rubbers, thermoplastic polyolefin elastomers, thermoplastic vulcanates, and thermoplastic polyurethanes, thermoplastic polyester, thermoplastic co-polyester, thermoplastic polyamides, and thermoplastic co-polyamides.

15. The medical device of claim 9, wherein the inflatable balloon has a wall thickness of about 0.008 inches to about 0.025 inches.

16. The medical device of claim 9, wherein a deployment pressure of the inflatable balloon is less than 10 psi.

17. The medical device of claim 9, wherein a burst pressure of the inflatable balloon is greater than 18 psi.

18. An elastomeric composition comprising:
at least one strain-induced crystallizable polymer; and
at least one amorphous polymer;
wherein the elastomeric composition has a Shore A hardness of 40 or less.

19. The elastomeric composition of claim 18, wherein the at least one strain-induced crystallizable polymer and the at least one amorphous polymer are styrene block polymers.

20. The elastomeric composition of claim 18, wherein the at least one strain-induced crystallizable polymer has a Shore A hardness of 30 or less.

21. The elastomeric composition of claim 19, wherein the at least one amorphous polymer has a Shore A hardness of 30 or less.

22. The elastomeric composition of claim 18, wherein the at least one strain-induced crystallizable polymer is provided in the polymer blend in an amount of about 30% to about 50% by weight.

23. The elastomeric composition of claim 18, wherein the at least one amorphous polymer is provided in the polymer blend in an amount of about 50% to about 70% by weight.

24. The elastomeric composition of claim 18, wherein the at least one strain-induced crystallizable polymer is a styrene-ethylene/butadiene-styrene elastomer (SEBS).

25. The elastomeric composition of claim 18, wherein the at least one amorphous polymer is one or both of an SIS or SIBS.

26. The elastomeric composition of claim 18 comprising 40 wt. % to 60 wt. % SIS and 60 wt. % to 40 wt. % SEBS.

27. The elastomeric composition of claim 18, wherein the elastomeric composition has an elongation at break of at least 500% according to ASTM D412.

28. The elastomeric composition of claim 18, wherein the elastomeric composition has a tensile stress at break of at least 400 psi according to ASTM D412.

29. The elastomeric composition of claim 18, wherein the elastomeric composition has a tensile modulus lower than 200 psi according to ASTM D638.

30. The elastomeric composition of claim 18, wherein the elastomeric composition excludes latex materials, natural rubbers, thermoplastic polyolefin elastomers, thermoplastic vulcanates, and thermoplastic polyurethanes, thermoplastic polyester, thermoplastic co-polyester, thermoplastic polyamides, and thermoplastic co-polyamides.

31. The elastomeric composition of claim 18, wherein the elastomeric composition has a Shore A hardness of 30 or less.

32. A medical device comprising:
a delivery catheter; and
an inflatable balloon secured to the delivery catheter, the inflatable balloon comprising the elastomeric composition of claim 18.

33. A method of manufacturing a balloon for a balloon catheter, the method comprising:
melting one or more thermoplastic elastomers;
extruding the one or more thermoplastic elastomers to form an extruded tube; and
mounting the extruded tube onto a catheter;
wherein the extruded tube has a Shore A hardness of 40 or less.

34. The method of claim 33, wherein the extruded tube has a Shore A hardness of 30 or less.

35. The method of claim 33, further comprising cutting the extruded tube to a desired length before the mounting.

36. The method of claim 33, further comprising stretching the extruded tube during the mounting.

37. The method of claim 36, wherein the mounting is achieved by heat, adhesive or by winding filaments over the extruded tube at proximal and distal ends of the extruded tube.

38. The method of claim 33, wherein the method excludes one or more of the following steps: dip molding, vulcanizing, blow molding, and annealing.
